(19) United States
(12) Patent Application Publication
(10) Pub. No.: US 2011/0144516 A1
(43) Pub. Date: Jun. 16, 2011

(54) RELATIVE CONTRIBUTION OF THORACIC MUSCLES TO BREATHING

(76) Inventors: Joseph Fisher, Thornhill (CA); Alex Vesely, Vancouver (CA); Dahlia Balaban, Toronto (CA); James Duffin, Toronto (CA); Greg D. Wells, Toronto (CA)

(21) Appl. No.: 12/992,837

(22) PCT Filed: May 15, 2009
(86) PCT No.: PCT/CA2009/000679

§ 371 Date: Feb. 22, 2011

Related U.S. Application Data
(60) Provisional application No. 61/053,938, filed on May 16, 2008.

(51) Int. Cl. A61B 5/08

(52) U.S. Cl. 600/529

(57) ABSTRACT

A method and apparatus for evaluating the relative contribution of the diaphragm versus other thoracic muscles to breathing by obtaining measurements of parameters that correlate with changes in thoracic cavity and intra-abdominal cavity pressures over identical time increments and organizing the measurement data in manner that reveals whether the pressures changes are characteristic of the pattern of contemporaneous pressure changes in those cavities that accompany contraction and/or relaxation of the diaphragm muscles or whether the pressure changes are characteristic of the pattern of contemporaneous pressure changes in those cavities that accompany contraction and/or relaxation of the intercostal muscles.

### CROSS REFERENCE TO RELATED APPLICATIONS

This application is a national phase filing, under 35 U.S.C. §371(c), of International Application No. PCT/CA2009/000679, filed May 15, 2009, the disclosure of which is incorporated herein by reference in its entirety, which application claims the benefit, under 35 U.S.C. §119(e), of co-pending U.S. Provisional Application No. 61/053,938, filed May 16, 2008, the disclosure of which is incorporated in its entirety.

### FIELD OF THE INVENTION

The present invention relates to a method and apparatus for evaluating the relative contribution of thoracic and diaphragmatic muscles to breathing.

### BACKGROUND OF THE INVENTION

The chest can be viewed as a cone, where the neck is at the tip and a muscle called the diaphragm, which separates the chest from the abdominal contents, is at the base. When relaxed, the diaphragm is dome-shaped and bulges into the chest cavity. The diaphragm is very important in the process of breathing, which consists of inspiration (breathing in) and expiration (breathing out). At rest, the diaphragm is the primary muscle of inspiration. When it contracts, it flattens out and displaces the abdominal contents from the chest into the abdomen to increase the chest volume so as to allow the entrainment of air.

Sometimes, such as during exercise, the demand for gas exchange is increased. This can be achieved through increases in the minute volume of breathing, by increasing breathing frequency as well as increasing the size of the breaths. Increasing the size of the breath above what can be provided by contraction of the diaphragm can be accomplished by the contraction of the external intercostal muscles and the accessory muscles of respiration. The external intercostals are attached to the ribs, which are hinged such that when these muscles contract, the ribs are pulled out and the volume of the chest increases. This increase in chest volume decreases intra thoracic pressure below that of the atmosphere, and air is passively drawn into the chest through the mouth and nose. These intercostal muscles are recruited at large ventilatory loads but play a minor role in breathing at rest.

Many conditions, such as pneumonia, heart failure, obstructive lung diseases like asthma, restrictive lung diseases, sepsis, and neuromuscular disorders, result in ventilatory distress. This can sometimes be treated conservatively, such as with medication and oxygen, but in some cases these treatments are not sufficiently able to provide adequate volumes of air to maintain oxygenation and eliminate carbon dioxide, and ventilatory assistance must be provided in order to prevent patient death. Ventilatory assistance is usually provided by applying positive pressure during inspiration to offload the inspiratory efforts. The motive force providing intermittent positive pressure can be mechanical or manual. The interface between the device providing the assistance and the patient can be “noninvasive” (a mask strapped to the face) or invasive (an endotracheal tube placed in the trachea). Noninvasive methods are suitable for short term use (minutes to hours), while all long term (days to months) ventilatory support requires invasive means. There are patients that are clearly able to maintain the ventilation they require without becoming fatigued. There are also patients who have clearly passed the point of fatigue and are unable to generate the breathing level that they need to sustain life. However, there are many patients who are difficult to categorize as to their ventilatory status on clinical grounds. There are currently no objective ways to make that determination.

If long term assisted mechanical ventilation is required, it is similarly very difficult to predict who will be able to be weaned and breathe on their own and when it will be possible to wean them. Failure after an attempt at weaning from a mechanical ventilator will require re-insertion of an endotracheal tube, which usually results in major set-back for the patient and is an indicator of poor prognosis. This is often avoided by prolonging the period of ventilatory assistance. This is a problem because prolonged ventilatory support is resource intensive (very expensive). Furthermore, the longer ventilator support is maintained, the harder it is to wean the patient from the support.

An important predictor of successful weaning from a ventilator would be the quantification of the diaphragm's contribution to ventilation. The diaphragm is a striated skeletal muscle that is designed to work intermittently, under conditions of low resistance, for an indefinite period of time. The other inspiratory muscles, on the other hand, can work only for short periods of time before they fatigue. Therefore, if the diaphragm can no longer be the primary organ of ventilation, the capacity of the body to sustain ventilation becomes markedly limited.

The contribution of the diaphragm to ventilation is difficult to ascertain. Clinically, one can watch the movement of the patient's abdomen on inspiration; because the diaphragm moves into and compresses the abdominal cavity when it contracts, one can assume it is contributing to ventilation if the abdomen appears to bulge out on inspiration. In practice, however, this is difficult to assess. For example, abdominal movements in obese patients may be masked, patients with abdominal incisions may be reluctant to move their abdomens, and chest movements may obscure simultaneous abdominal movements. Fluoroscopy is a kind of moving X-ray that can be used to see diaphragmatic movement, but it is very difficult to perform, as it requires highly specialized equipment and the fluoroscopic signs are very subtle. Electromyography (EMG) of the diaphragm is possible, but it requires the placement of needles through the chest and lungs into the diaphragm and is therefore not practical for clinical use. Even if EMG signals are obtained, these imply attempts at contraction and do not necessarily imply the effectiveness of the diaphragm at increasing chest volume. Another possible method is to occlude the breathing passage, ask the patient to make a maximal inspiratory effort, and measure the generated pressure. This correlates to some extent with inspiratory reserve, but is not specific to the diaphragm. There is therefore a need for a method and apparatus that quantifies the contribution of the diaphragm to breathing that can be used to make clinical decisions regarding timing of implementing mechanical ventilatory support or ability to wean patients from ventilatory support.

### SUMMARY OF THE INVENTION

The present invention is directed a method and apparatus for evaluating the relative contribution of the diaphragm vs. other thoracic muscles (primarily the intercostal muscles) to breathing. The method involves determining whether an indicator of pressure change in the thoracic cavity is in phase with an indicator of pressure change in the abdominal cavity (for example, a pressure decrease in one is accompanied by a contemporaneous pressure decrease in the other) or out of phase (for example, pressure in the thoracic cavity is down while pressure in the abdominal cavity is up). A decrease in thoracic pressure accompanied by an increase in abdominal pressure will indicate that diaphragmatic muscles dominantly contribute to the intra-thoracic pressure declination because contraction of the diaphragm is expected to be manifested by an increase in abdominal pressure. On the other hand, where a declination in pressure, for example, in the thoracic cavity, is under the dominant influence of the intercostal muscles, it will not be accompanied by a rise in abdominal pressure; rather by a decrease in abdominal pressure because the contraction of the intercostals muscles will draw the diaphragm upward, reducing pressure in the abdominal cavity.

According to one embodiment of the invention, one way of assessing a change in thoracic cavity pressure is to monitor a volumetric change in the thoracic cavity, as such changes are negatively correlated with changes in thoracic pressure. According to one embodiment of the invention a volumetric change is recorded by monitoring the tidal volume of a patient's breath, which is in an indirect indicator of expansion and contraction accompanying inspiration and expiration, respectively. For example, an increase in tidal volume (indicating a decrease in intra-thoracic pressure resulting from expansion of the chest during inspiration) may be recorded to be out of phase with abdominal pressure which if recorded to increase, will, as stated above, indicates that diaphragmatic muscles dominantly contribute to chest expansion inasmuch as contraction of the diaphragm is expected to be manifested in an increase in abdominal pressure. If expansion of the thoracic cavity (resulting in a decrease in intra-thoracic cavity pressure), on the other hand, is under the dominant influence of the intercostal muscles, it will be accompanied by a concomitant decrease (in phase) in abdominal pressure.

According to one embodiment of the invention, a record of an increase or decrease in abdominal pressure will be obtained by measuring intra-gastric pressure in real time, for example using an intra-gastric tube, such as, a tube of the type used to infuse fluid feeds or aspirate stomach contents in a patient, the proximal end of the tube (typically outside the patient) may be attached to pressure transducer connected to a microprocessor that records the pressure as a function of time. Recording tidal volume over the same increments in time will enable the microprocessor to provide a surrogate indication of phase relationship between intra-abdominal pressure and phase of ventilation. Alternatively, the flux in pressure in the abdominal cavity, up or down, may be recorded by measuring intra-bladder pressure, which may be convenient if the patient has been catheterized but does not have an intra-gastric tube in place.

According to another embodiment of the invention pressure in the abdominal cavity can be measured directly from an intra-gastric balloon, or a tube in the stomach and pressure in the thoracic cavity may be measured in real time by recording intra-esophageal pressure in real time. Recording both the intra-abdominal pressure and intra-thoracic pressure will enable the microprocessor to provide a read out of the phase relationship between intra-abdominal pressure and intra-thoracic pressure. When the intra-abdominal pressure is 180 degrees out of phase with the intra-thoracic pressure the diaphragm is the predominant organ of ventilation. When the pressures in the abdominal cavity and the thoracic cavity are in phase (0 degree phase shift) the thoracic muscles dominate ventilation and the diaphragm makes a minor, if any, contribution to air movement.

The invention is also directed to an apparatus for recording the relative contribution to breathing of the diaphragm and intercostal muscles comprising:

1) means for monitoring for a series of breaths or common increments in real time:

the value of a first variable correlated with intra-thoracic pressure;

the value of a second variable correlated with intra-abdominal pressure;

machine intelligence outputting the relative direction of flux of the monitored variables.

The term “outputting the relative direction of flux” means generating output that enables a user to discern whether the change in the respective values of the variables over successive breaths or increments in time are in or out phase with one another. The output is optionally generated on a display, on a printout or on a machine-readable data storage device.

The term “inadequate” or “adequate” when used to describe the contribution of the diaphragm to breathing means a predictable (based on a medically acceptable margin of safety) inability or ability (respectively) of the patient to indefinitely sustain adequate minute ventilation to meet metabolic demand.

According to one embodiment of the invention, the invention is directed to an apparatus comprising: (1) a monitor for delimiting in time at least the approximate beginning and end of an inspiratory phase of breathing, optionally, for multiple breaths, optionally for a series of breaths, optionally throughout both the inspiratory and expiratory phases of breathing. This monitor may be for quantitative or qualitative monitoring, for example by a measurement device, optionally a device for measuring the volume and/or direction of flow of gas in or out of the lungs during that monitored time, for example a pneumotachograph or means for measuring the value of variable correlated with pressure in the thoracic cavity, for example a pressure measurement device, for example, a pressure measurement device for measuring esophageal pressure, for example, a pressure transducer. This pressure transducer is optionally used for measuring intra-esophageal pressure; (2) a monitoring device for monitoring a variable correlated with intra-abdominal pressure that is not masked by other variables, optionally the direction of flux of this correlate, optionally a quantifiable variable e.g. one that can be measured, optionally gastric pressure, optionally a pressure measurement device, for example a second pressure transducer, optionally operatively connected to a tube for measuring intra-gastric pressure; (3) and means for associating the phase of breathing, for example via recording a variable correlated with intra-thoracic pressure, for example, the volume of air entering and leaving the lung, with a direction of change in pressure in the abdominal cavity, for example a microprocessor programmed to generate a record of the direction of flux of the monitored variable (e.g. the measured intra-gastric pressures) for the monitored period, for example for correlating the instantaneous pressures with the period spanning at least the inspiratory phase and preferably the expiratory phase of breathing, preferably for multiple breaths, optionally for a series of breaths.

According to one embodiment, the intra-abdominal and intra-thoracic pressures are measured by pressure transducers of the type adapted to be used with an intra-gastric/esophageal tube, optionally fitted with a small balloon at the distal end to prevent occlusion of the lumen of tube by contact with the gastric or esophageal tissues. Optionally, the microprocessor associates and preferably records the respective intra-esophageal and intra-gastric pressure values for inspiratory and expiratory segments of respective breaths and/or for common time increments and generates output that enables the user to discern a consistent pattern of directional changes of both variables relative to a baseline value that enables a user to discern whether the change in the respective values of the variables over successive breaths or increments in time are in or out phase with one another.

According to one embodiment of the invention introduced above, the invention is directed to an apparatus comprising a device 32 for measuring tidal volume in real time; a pressure transducer 10 for measuring gastric pressure in real time and a microprocessor 16 programmed to generate a record of the relative direction of flux of the measured values. Optionally, the microprocessor 16 associates and record the respective tidal volumes and intra-gastric pressure values in respective breaths or common time increments and generates output, optionally in the form of a plot of tidal volume and gastric pressures over time that enable the user to visualize a distinct pattern of loops in that indicate whether abdominal and intra-thoracic pressure are in or out phase with one another.

According to one embodiment of the invention, introduced above, the variable correlated with intra-abdominal pressure is gastric pressure. According to one embodiment of the invention, gastric pressure is measured with a device comprising a pressure transducer. The pressure transducer is of the type adapted to measure pressure using a tube inserted into the stomach. According to another embodiment of the invention the variable correlated with intra-abdominal pressure is bladder pressure. According to one embodiment of the invention introduced above, the variable correlated with intra-thoracic pressure is tidal volume which may be measured means by measuring airflow at a patient airway interface. According to one embodiment of the invention, the variable correlated with intra-thoracic pressure is intra-esophageal pressure which may be measured with a pressure transducer for recording pressure via a tube inserted into the esophagus.

According to another aspect of the invention, machine intelligence is used to plot a graph of tidal volume on one axis and gastric pressure on the other axis on a breath by breath basis so as to record distinct tracings that can be differentiated by a technician, for example loop-like tracings that can be discriminated by eye when the relative contribution of the diaphragm and intercostals muscles to breathing is dominated by one or the other group of muscles. Therefore in a broader aspect the invention is directed to a form of machine intelligence, optionally in the form of a computer program or a processor programmed to correlate the inspiratory phase of breathing with a direction of flux in abdominal pressure, optionally with measurements of a variable correlated with abdominal pressure, optionally in which the phase of breathing is represented by measurements of airflow or intra-thoracic pressure (optionally intra-esophageal pressure), wherein a positive or inverse correlation or a phase relationship can be quantified or visualized.

According to one embodiment of the invention, a bi-lumen tube may be used to record pressure in the esophagus and stomach contemporaneously. Optionally, the distal end of one or both tubes is fitted with a balloon to prevent occlusion of the tube, for example via contact with the esophagus or stomach respectively. Optionally, a tri-lumen tube additionally comprises a gastric feeding tube.

### BRIEF DESCRIPTION OF THE DRAWINGS

FIG. 1 is a schematic representation of one embodiment of an apparatus according to the invention.

FIG. 2a is a graph of measured intra-gastric pressure versus time juxtaposed with a graph of measured intra-esophageal pressure versus time, in which the same time points are aligned.

FIG. 2b is a graphical representation in the form of a plot of measured tidal volume (V) (volume inspired and expired relative to what is already in the lung) over time (increasing upon inspiration and decreasing again upon expiration) versus measured gastric pressure (Pg).

FIG. 3a is a contrasting graph of intra-gastric pressure versus time juxtaposed with a graph of intra-esophageal pressure versus time, in which the same time points are aligned. This graph was generated under conditions described in connection with FIG. 3b below.

FIG. 3b is a graphical representation of measured tidal volume (V) over time plotted again gastric pressure (Pg). V and Pg were measured under conditions in which the flow of gas (oxygen enriched air) made available to the subject for breathing was reduced to force the subject to re-breathe the subject's expired gas from a previous breath thus inducing the patient to increase their inspiratory effort, in particular, in terms of tidal volume, in subsequent breaths.

FIG. 4a is a contrasting graph of measured intra-gastric pressure versus time juxtaposed with a graph of measured intra-esophageal pressure versus time, in which the same time points are aligned. This graph was generated under conditions described in connection with FIG. 4b below.

FIG. 4b is a graphical representation in the form a plot of measured tidal volume (V) over time versus measured gastric pressure (Pg). V and Pg were measured under conditions in which the flow of gas (oxygen enriched air) made available to the subject for breathing was further reduced to force the subject to re-breathe the subject's expired gas from a previous breath thus inducing the patient to further increase their inspiratory effort, in particular, in terms of tidal volume, in subsequent breaths relative to conditions under which the data in FIGS. 3a and 3b was gathered.

FIG. 5a is a contrasting graph of measured intra-gastric pressure versus time juxtaposed with a graph of measured intra-esophageal pressure versus time, in which the same time points are aligned. This graph was generated under conditions described in connection with FIG. 5b below.

FIG. 5b is a graphical representation of measured tidal volume (V) over time versus gastric pressure (Pg). V and Pg were measured under conditions in which the flow of gas (oxygen enriched air) made available to the subject for breathing was still further reduced to force the subject to re-breathe the subject's expired gas from a previous breath thus inducing the patient to further increase their inspiratory effort, in particular, in terms of tidal volume, in subsequent breaths relative to conditions under which the data in FIGS. 4a and 4b was gathered.

### DETAILED DESCRIPTION OF THE INVENTION

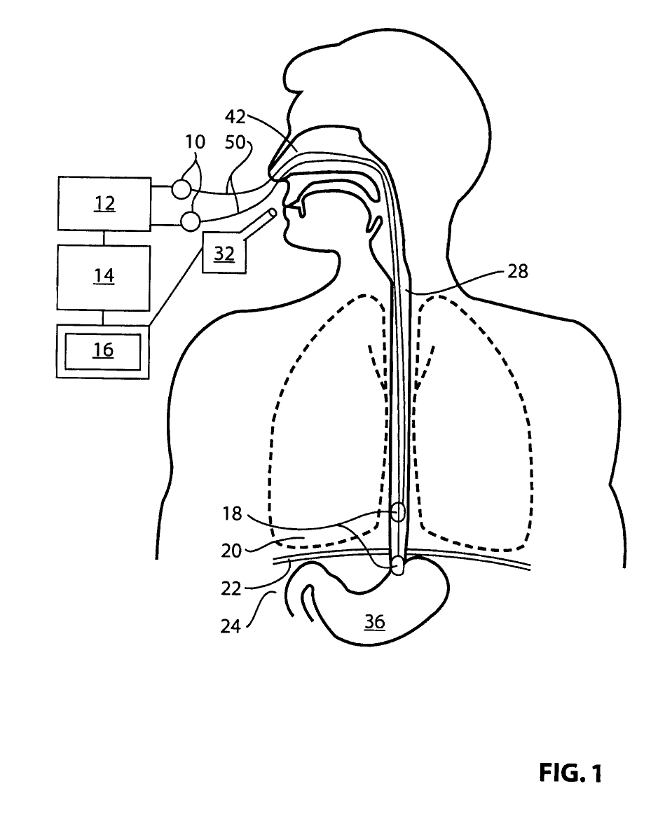

According to one embodiment of the invention, gastric pressure can be used to estimate intra-abdominal pressure. Optionally, as shown FIG. 1, gastric pressure is obtained by passing a tube 50 through the mouth or nasal cavity 42 and directing it into the stomach 36. These types of tubes are commercially available as “pediatric feeding tubes”, “naso-gastric tubes”, “gastrostomy tubes”, “esophageal balloons”, “gastric balloons” and others. Once the distal end of the tube is in the stomach 36, the pressure in the stomach can be measured by attaching the proximal end of the tube to a pressure transducer 10.

In one embodiment, bladder pressure can be used to estimate intra-abdominal pressure. This can be optionally accomplished by using an in-dwelling catheter to measure pressure across the bladder wall.

There are many optional devices to measure tidal volume, including “mass flow sensors”, “pneumotachographs”, “turbines”, and Pitot tubes.

In, one embodiment of the invention, air flow in and out of the lung (tidal volume) is plotted against gastric pressure on a breath by breath basis. This results in a graphical representation in the form of a loop or loops. The contribution of thoracic (intercostal and accessory) muscles is represented by one loop (thoracic loop). The contribution of the diaphragm is represented by a separate, but connected, loop (diaphragmatic loop). Thus the contribution, if any, of the diaphragm can be identified by examining the slope of the axis of the diaphragmatic loop.

The method allows identification of a sustainable, diaphragm-predominant, state of drawing air in and out of the lungs. Furthermore, the method can identify an unsustainable, accessory muscle-dependent, breathing pattern through the observation of the gastric pressure vs. tidal volume loops. Therefore the present invention can optionally be used to determine whether a ventilated patient should be removed from a ventilator to breathe on their own. The patient already has a naso-gastric tube in situ that is used for feeding and decompressing the stomach. If this naso-gastric tube is attached to a transducer and, optionally, a pneumotachograph is attached to the endotracheal tube (not shown), the patient could be allowed to begin to make spontaneous ventilatory efforts while the loops are observed. If they have, or develop over time, the diaphragmatic loop, it can be assumed that the patient will be able to recruit the diaphragm. If only thoracic muscle loops are present, it can be assumed that the diaphragm may not be able to be recruited and the patient may fail weaning from the ventilator. The use of the ventilator would then be resumed with little distress to the patient.

### Bladder pressure can be optionally measured using two three-way stopcocks attached serially to a pressure transducer as described in Cheatham M L, Safcsak K. Intraabdominal pressure: a revised method for measurement. J Am Coll Surg 1998; 186(5):594-595. The first stopcock is connected to a source of normal saline and the second stopcock (pressure transducer proximal) is connected to a Luer lock syringe. Briefly, an infusion catheter is inserted into the urinary drainage tubing and subsequently attached to the first stopcock using pressure tubing. The urinary drainage tubing is clamped in a position immediately distal to the catheter. A total of 100 mL of normal saline is then instilled into the bladder. After ensuring all air is removed from the urinary catheter, the patient's intra-abdominal pressure is measured using the pressure transducer.

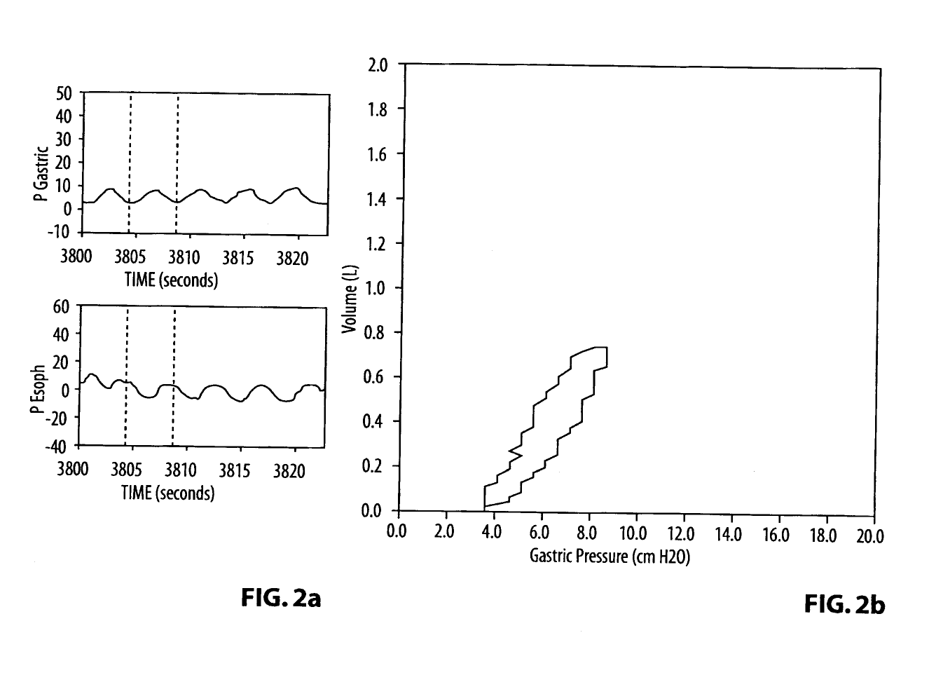

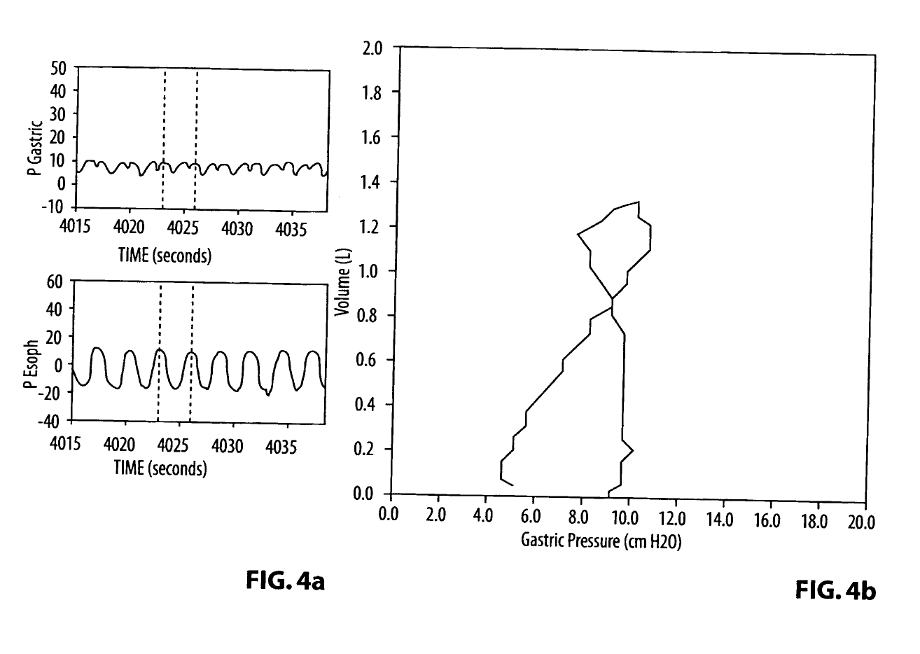

As shown in FIGS. 2-5, the plotting of volume versus intra-abdominal pressure results in a series of characteristic loops. Tidal volume is optionally obtained by mass flow sensors, pneumotachographs, turbines, or Pitot tubes. In this embodiment, intra-abdominal pressure is estimated using gastric pressure measurements. As shown in FIG. 1, pressure in the abdominal cavity 24 is optionally ascertained by measuring intra-gastric pressure which is optionally obtained by passing a tube 50 (optionally connected to a balloon 18 to prevent occlusion of the distal end of the tube) through the nasal cavity 42 or mouth, into the stomach 36 and attaching a pressure transducer 10 to the proximal end of the tube. The pressure transducer 10 is optionally connected to an amplifier 12 and then to a microprocessor or computer, 16, in this embodiment via an analog to digital converter 14. Optionally, pressure measurements in the thoracic cavity 20 may be measured by a similar tube 50 and balloon 18 lowered in the esophagus 28 and connected at its proximal end to another pressure transducer 10. A processor, for example a computer and computer program, are then optionally used to analyze pressure and volume data and, in the case of volume (litres) v. pressure (cm of H₂O), to convert them into visual graphs in the form of loops.

FIG. 2a illustrates a graph of continuous pressure measurements (Y axis—cm of H₂O) recorded in both the stomach and esophagus (of a subject that is able to recruit the diaphragm normally) as a function of time (in seconds) supplemented for emphasis with two vertical lines to demarcate a segment of the pressure wave that corresponds to a single breath, beginning with an inspiratory phase, and followed by an expiratory phase. As demonstrated in the graphs an initial pressure rise in the stomach is followed by a pressure decline, and is matched, out of phase, with an initial pressure decline followed by a pressure rise in the esophagus. This is what is expected when a subject inspires by recruiting the diaphragm. Accordingly, the direction of change in terms of the inspiratory phase is a decline, more particularly a steady decline i.e. an increasing pressure drop in the thoracic cavity, measured in terms of esophageal pressure, which if followed, is reversed, again, and is characteristically a steady incline back to a similar value. This is accompanied, directionally speaking, by a concomitant pressure rise during inspiration in the abdominal cavity measured in terms of gastric pressure. The respective changes in pressure in both cases are then reversed upon expiration and reach their approximate original values. In terms of an increase (inspiration) and subsequent decrease (expiration) in volume (corresponding to first a decrease in pressure and the a return to an approximate previous pressure value), the resulting loop illustrates a pattern including a positive slope that is characteristic of the pattern of contemporaneous pressure changes in the respective thoracic and abdominal cavities that accompany contraction and/or relaxation of the diaphragm muscles, with an approximate return to original values for the next breath.

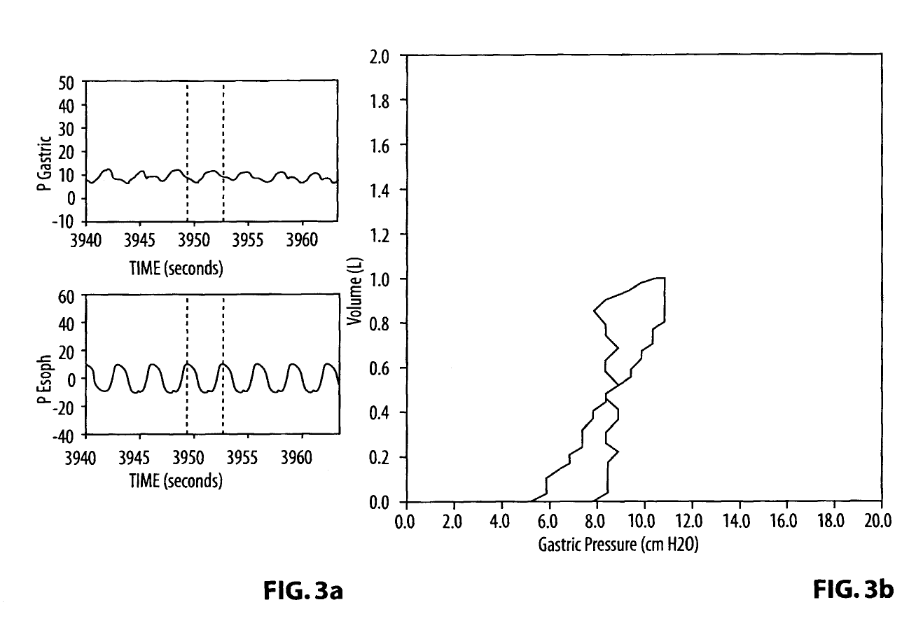

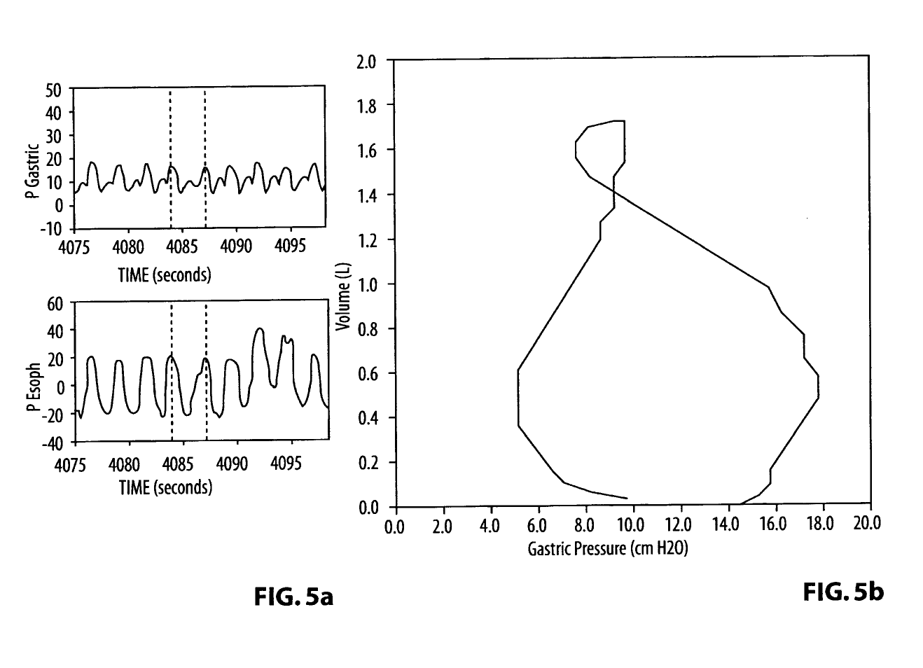

Data for FIGS. 3-5 was obtained from a subject who was asked to breath via a sequential gas delivery circuit or re-breathing circuit (not shown) of the type illustrated in FIGS. 2 and 5 of WO2004/073779. This experiment was designed to illustrate the loops of various patterns accompanying a changing relative contribution of the diaphragmic and thoracic muscles brought about when the subject is forced to rebreath expired gas (relatively high in CO₂ content). This was done in order to impose upon the subject (by increasingly reducing the flow of fresh gas), conditions under which the subject would have to breathe incrementally more deeply (resulting, in particular, in an increase in tidal volume) so the contribution of the thoracic muscles would become increasingly greater to achieve this increasing tidal volume.

The pressure waves in FIG. 3a are not so distinctly out of phase. The loop in FIG. 3b represents a greater contribution of the work of the intercostal and accessory muscles to the greater inspiratory effort and tidal volume demonstrated by component parts of volume/pressure tracing. The initial pressure value is not recovered within the breath. The greatest characteristic change in terms of wave phase alignment (FIG. 2a) and characteristic loop in FIG. 2b is shown in FIGS. 5a demonstrating the pressure waves are at least initially in phase and FIG. 5b where an increasing inspired volume is accompanied by an at least initial concomitant drop in the pressure of abdominal cavity showing that the diaphragm is being drawn up by the action of the accessory muscles rather than being flattened down into the abdominal cavity to increase intra-abdominal pressure were it to contract. Therefore only a small pressure increase following the initial pressure decrease is evident before the tidal volume starts to a reach its highest level. Thus the contribution of the thoracic muscles and diaphragm can be identified by examining the slope of the axis of the loop, thereby attributing the ventilation to the appropriate muscle group.

Therefore, as shown in FIG. 2-5, inspiratory phase and optionally expiratory phase specific qualitative variables (in terms of the thoracic cavity—at least the breathing phase and in terms of the abdominal cavity at least the direction of flux), optionally, corresponding (concomitant) directions of flux for both cavities, optionally quantified variables for at least the abdominal cavity (volume or pressure are herein demonstrably useful as quantified variables for the thoracic cavity) in the form of pressure correlated measurements may be processed by a microprocessor or computer to generate user friendly output evidencing the relative direction of flux of the monitored variables

We claim:

1. A method for evaluating the relative contribution of the diaphragm versus other thoracic muscles to breathing:
obtaining measurements of parameters that correlate with changes in thoracic cavity and intra-abdominal cavity pressures over identical time increments; and
organizing the measurement of data in manner that outputs at least one of the following:
a) whether the pressures changes are characteristic of the pattern of contemporaneous pressure changes in those cavities that accompany contraction and/or relaxation of the diaphragm muscles;
b) whether the pressure changes are characteristic of the contemporaneous pattern of pressure changes in those cavities that accompany contraction and/or relaxation of the thoracic muscles.

2. A method according to claim 1, wherein the change in thoracic cavity pressure is represented and thereby quantified by a volumetric change in the thoracic cavity.

3. A method according to claim 2, wherein the volumetric change in the thoracic cavity is measured by the tidal volume of a patient's breath.

4. A method according to claim 1, wherein the intra-thoracic pressure is indirectly measured in real time by monitoring intra-esophageal pressure in real time.

5. A method according to claim 4, wherein the intra-esophageal pressure is measured using an intra-gastric tube fitted with a pressure transducer.

6. A method according to claim 1, wherein the intra-abdominal pressure is obtained by measuring intra-gastric pressure in real time.

7. A method according to claim 6, wherein intra-gastric pressure is measured using an intra-gastric tube.

8. A method according to claim 7, wherein intra-gastric pressure is measured using an intra-gastric tube of the type normally used to feed a ventilated patient.

9. A method according to claim 7, wherein the proximal end of the tube is attached to a pressure transducer connected to a microprocessor which records the pressure as a function of time.

10-11. (canceled)

12. A method according to claim 3, wherein machine intelligence is used to plot a graph of tidal volume versus intra-gastric pressure on a breath by breath basis to record distinct tracings that can be differentiated by a technician when the relative contribution of the diaphragm and intercostal muscles to breathing is dominated by one or the other group of muscles, optionally the tracings are loop-like tracings that can be discriminated by eve.

13. (canceled)

14. An apparatus for recording the relative contribution of the diaphragm versus other thoracic muscles to breathing, the apparatus comprising:
a means for monitoring (optionally a measurement device) in a series of breaths or common increments in real time:
the value of a first variable correlated with intra-thoracic pressure
the value of a second variable correlated with intra-abdominal pressure
machine intelligence outputting the relative direction of flux of the monitored variables.

15. An apparatus according to claim 14, wherein the output is generated on a display, printout or machine-readable data storage device.

16. An apparatus according to claim 14, wherein the apparatus comprises a pressure transducer for measuring intra-esophageal pressure; a pressure transducer for monitoring intra-gastric pressure and a microprocessor programmed to record the relative direction of flux of the measured pressures, optionally the pressure transducers are of the type adapted to be used with an intra-gastric or intra-esophageal tube, optionally a bi-lumen tube is used to simultaneously record pressure in the esophagus and stomach, optionally, a tri-lumen tube is used comprising a gastric feeding lumen and respective lumens to record pressure in the esophagus and stomach simultaneously, optionally an intra-gastric or intra-esophageal tube is fitted with a small balloon at the distal end, optionally the microprocessor generates an output enabling the user to discern a consistent pattern of directional changes of both variables relative to a baseline value, optionally the microprocessor associates and records intra-esophageal and intra-gastric pressure values for inspiratory and expiratory segments of respective breaths and/or for common time increments

17-19. (canceled)

20. An apparatus according to claim 14, wherein the apparatus comprises a device for measuring tidal volume in real time; a pressure transducer for measuring intra-gastric pressure in real time and a microprocessor programmed to generate a record of the relative flux of the measured values.

21. An apparatus according to claim 20, wherein the microprocessor associates and records tidal volume and intra-gastric pressure values for inspiratory and expiratory segments of respective breaths and/or for common time increments.

22. An apparatus according to claim 20, wherein the microprocessor generates an output enabling the user to discern a consistent pattern of directional changes of both variables relative to a baseline value, optionally the microprocessor output is in the form of a plot of tidal volume and gastric pressures over time, enabling the user to visualize a distinct pattern of loops indicating whether intra-abdominal and intra-thoracic pressures are in or out of phase with one another.

23-25. (canceled)

26. An apparatus for recording the relative contribution of the diaphragm versus other thoracic muscles to breathing comprising a monitor for delimiting in time at least the approximate beginning and end of an inspiratory phase of breathing, a monitoring device for monitoring a variable correlated with intra-abdominal pressure, and means for associating at least the inspiratory phase of breathing, with a direction of change in pressure in the abdominal cavity for a time span spanning at least the inspiratory phase of breathing.

27. An apparatus according to claim 26, wherein the time span covers a full inspiratory and expiratory phase of one breath or full inspiratory and expiratory phases of a series of breaths.

28. (canceled)

29. An apparatus according to claim 26, wherein the phase of breathing is monitored by monitoring a variable correlated with intra-thoracic pressure, optionally intra-esophageal pressure.

30. (canceled)

31. An apparatus according to claim 29, wherein the means for monitoring the phase of breathing is a device for measuring a pressure correlated with intra-thoracic pressure.

32. An apparatus according to claim 26, wherein the means for monitoring the phase of breathing is a means for measuring the volume change (increase/decrease) and/or direction of flow of gas in or out of the lungs.

33. An apparatus according to claim 26, wherein the monitoring device for monitoring a variable correlated with intra-abdominal pressure is a pressure measurement device, optionally the pressure measurement device is adapted to measure intra-gastric pressure.

34. (canceled)

35. An apparatus according to claim 26, wherein means for associating at least the inspiratory phase of breathing, with a direction of change in pressure in the abdominal cavity is a means for organizing the data in manner that reveals at least one of the following:
c) whether the pressures changes are characteristic of the pattern of contemporaneous pressure changes in those cavities that accompany contraction and/or relaxation of the diaphragm muscles;
d) whether the pressure changes are characteristic of the contemporaneous pattern of pressure changes in those cavities that accompany contraction and/or relaxation of the intercostals muscles.

36-38. (canceled)
